(19) United States
(12) Patent Application Publication
(10) Pub. No.: US 2022/0362525 A1
(43) Pub. Date: Nov. 17, 2022

(54) DEVICES AND METHODS FOR GUIDEWIRE MANAGEMENT

(71) Applicant: THE CHILDREN'S HOSPITAL OF PHILADELPHIA, Philadelphia, PA (US)

(72) Inventor: Mark WEBER, Philadelphia, PA (US)

(73) Assignee: THE CHILDREN'S HOSPITAL OF PHILADELPHIA, Philadelphia, PA (US)

(21) Appl. No.: 17/597,310

(22) PCT Filed: Jul. 9, 2020
(86) PCT No.: PCT/US20/41361

§ 371 Date: Jan. 3, 2022

Related U.S. Application Data
(60) Provisional application No. 62/872,061, filed on Jul. 9, 2019.

(51) Int. Cl. A61M 25/09; A61M 39/06; A61M 25/00

(52) U.S. Cl. CPC A61M 25/09041; A61M 39/06; A61M 25/0097

(57) ABSTRACT

Devices and methods for management of a guidewire during catheter insertion. Particular embodiments include unidirectional valves to allow movement of a guidewire in one direction only.

### CROSS-REFERENCE TO RELATED APPLICATIONS

This application claims priority to U.S. Provisional Patent Application Ser. No. 62/872,061 filed Jul. 9, 2019, the entire contents of which are incorporated herein by reference.

### BACKGROUND INFORMATION

The Seldinger technique, also known as Seldinger wire technique, is a medical procedure to obtain safe access to blood vessels and other hollow organs via a guidewire. It is named after Dr. Sven Ivar Seldinger (1921-1998), a Swedish radiologist who introduced the procedure in 1953.

During typical procedures, the desired vessel or cavity is punctured with a sharp hollow needle, with ultrasound guidance if necessary. A round-tipped guidewire is then advanced through the lumen of the needle, and the needle is withdrawn. A sheath or intravascular catheter can now be passed over the guidewire into the cavity or vessel. After passing a sheath or catheter, the guidewire is withdrawn [1].

Loss of the guidewire into the blood vessel is a significant complication [2]. Retained guidewires can lead to thromboembolism, cardiac and vascular perforation, arrhythmias and infection.

Approximately 5 million central venous catheters (CVC) are placed annually [3]. The National Quality Forum considers retained guidewires as a “never event”; however, it is estimated that the rate of inadvertent retained guidewires is 1 out of every 3000 CVC placements [4]. In one instance, a guidewire was found twenty years after CVC placement [5].

Accordingly, a need exists for devices and methods to reduce the risks of inadvertent retainment of guidewires in patients.

### SUMMARY

Exemplary embodiments of the present disclosure comprise devices and methods configured to manage a guidewire during placement of a catheter in a patient. Particular embodiments include a guidewire management device to help minimize the inadvertent loss of an intravascular guidewire during a central line or arterial line placement.

In specific embodiments, the device can be coupled to the distal end of a central catheter via a Luer connector, which is a standard fitting on catheters, syringes and intravascular equipment. In particular embodiments, the device is a small unit generally equivalent in size to a central line cap. During use the device will allow the guidewire to only pass in one direction, and not to inadvertently advance further into the vasculature of the patient. The use of restraining valves in the device will prevent the guidewire from inadvertently advancing into the patient during the insertion of the central line.

As explained further below, if the guidewire needs to be advanced for any reason, a user can compress (e.g. pinch or squeeze) the device to allow intentional free movement of the guidewire.

Certain embodiments include a device for guidewire management, where the device comprises: a housing comprising a first end and a second end; a central lumen extending through the housing from the, wherein the central lumen extends from the first end to the second end; and a unidirectional valve in the central lumen. In particular embodiments, the unidirectional valve is configured to allow movement of a guidewire in a first direction through the unidirectional valve; and the unidirectional valve is configured to prevent movement of a guidewire in a second direction through the unidirectional valve, wherein the second direction is opposite of the first direction.

Specific embodiments further comprise: a first threaded coupling in the central lumen, wherein the first threaded coupling is proximal to the first end; and a second threaded coupling in the central lumen, wherein the second threaded coupling is proximal to the second end, where: the first threaded coupling is configured to couple to a central venous catheter. In some embodiments, the first direction is from the first end toward the second end.

Certain embodiments further comprise a wall surrounding the central lumen, and wherein the unidirectional valve is angled from the wall toward the second end of the device. In particular embodiments, the unidirectional valve is angled from the wall at an angle between 100 and 120 degrees, and in specific embodiments the unidirectional valve is angled from the wall at an angle of approximately 109 degrees.

In some embodiments, the unidirectional valve comprises a distal end, and the distal end is spaced apart from the wall by a gap. In specific embodiments, the gap is less than 0.5 mm. In certain embodiments, the device is a unitary structure and the housing and the unidirectional valve is formed from the same material. In particular embodiments, the material is a polymer. In some embodiments, the material is an elastomer, and in specific embodiments the material is latex, silicone rubber, nylon, or polyurethane.

In certain embodiments, the unidirectional valve is configured as a flexible flap. In particular embodiments, the unidirectional valve is a first unidirectional valve, and the device further comprises a second unidirectional valve. In some embodiments, the second unidirectional valve is configured equivalently to the first unidirectional valve.

Certain embodiments include a method of inserting a catheter into a patient, where the method comprises: inserting a guidewire into a patient; obtaining a catheter comprising a proximal end and a distal end, wherein the catheter comprises a device according to the present disclosure (e.g. the device recited in claim 1) coupled to the distal end; and advancing the catheter along the guidewire toward the patient, wherein the guidewire extends through the catheter and the device according to the present disclosure. In particular embodiments, the method comprises inserting the catheter into the patient, and removing the guidewire from the device according to claim 1 and from the catheter.

In specific embodiments, the guidewire and the catheter are inserted into a vein of the patient. In certain embodiments, the guidewire is restricted from moving from the distal end of the catheter toward the patient when the guidewire is inserted through the device according to the present disclosure (e.g. the device recited in claim 1). Particular embodiments include further comprise compressing the device according to the present disclosure (e.g. the device recited in claim 1), where: the guidewire is permitted to move from the distal end of the catheter toward the patient while the device according to the present disclosure is compressed. In specific embodiments, the guidewire is inserted into the patient through a needle. In certain embodiments, the device according to the present disclosure (e.g. the device recited in claim 1) is coupled to the distal end of the catheter via a threaded coupling.

Particular embodiments include a system comprising: a catheter comprising a proximal end and a distal end; and a device coupled to the distal end, where the device is configured to allow movement of a guidewire in a first direction through the device and the catheter, and the device is configured to prevent movement of a guidewire in a second direction through the device and the catheter, wherein the second direction is opposite of the first direction. In specific embodiments, the device coupled to the distal end comprises: a housing comprising a first end and a second end; a central lumen extending through the housing from the, wherein the central lumen extends from the first end to the second end; and a unidirectional valve in the central lumen. In certain embodiments, the unidirectional valve is configured to allow movement of a guidewire in a first direction through the unidirectional valve; and the unidirectional valve is configured to prevent movement of a guidewire in a second direction through the unidirectional valve, wherein the second direction is opposite of the first direction.

As used herein, the term “valve” is not limited to devices or mechanisms that control or restrict fluids. The term “valve” as used herein includes devices and mechanisms that can control or restrict movement of a solid (including for example, a guidewire) passing through the device. In particular, a “unidirectional valve” as used herein includes devices and mechanisms that can allow a guidewire to move in one direction through the device, but prevent movement of the guidewire in the opposite direction.

In the following disclosure, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE DRAWINGS

The following drawings form part of the present specification and are included to further demonstrate certain aspects of the present disclosure. The invention may be better understood by reference to one of these drawings in combination with the detailed description of specific embodiments presented herein.

FIG. 1 is an orthogonal side view of an exemplary embodiment of the present disclosure.

FIG. 2 is a section view taken along line A-A of the embodiment of FIG. 1.

FIG. 3 is a first perspective view of the embodiment of FIG. 1.

FIG. 4 is a second perspective view of the embodiment of FIG. 1.

FIG. 5 is a third perspective view of the embodiment of FIG. 1.

FIG. 6 is a perspective view of a first step of a method of using the embodiment of FIG. 1.

FIG. 7 is a perspective view of a second step of a method of using the embodiment of FIG. 1.

FIG. 8 is a perspective view of a third step of a method of using the embodiment of FIG. 1.

FIG. 9 is a perspective view of a fourth step of a method of using the embodiment of FIG. 1.

FIG. 10 is a perspective view of a fifth step of a method of using the embodiment of FIG. 1.

FIG. 11 includes section and perspective views of a second exemplary embodiment of the present disclosure.

FIG. 12 includes section and perspective views of a third exemplary embodiment of the present disclosure.

FIG. 13 includes section and perspective views of a fourth exemplary embodiment of the present disclosure.

FIG. 14 includes section and perspective views of a fifth exemplary embodiment of the present disclosure.

FIG. 15 includes section and perspective views of a sixth exemplary embodiment of the present disclosure.

FIG. 16 includes section and perspective views of a seventh exemplary embodiment of the present disclosure.

### DETAILED DESCRIPTION OF ILLUSTRATIVE EMBODIMENTS

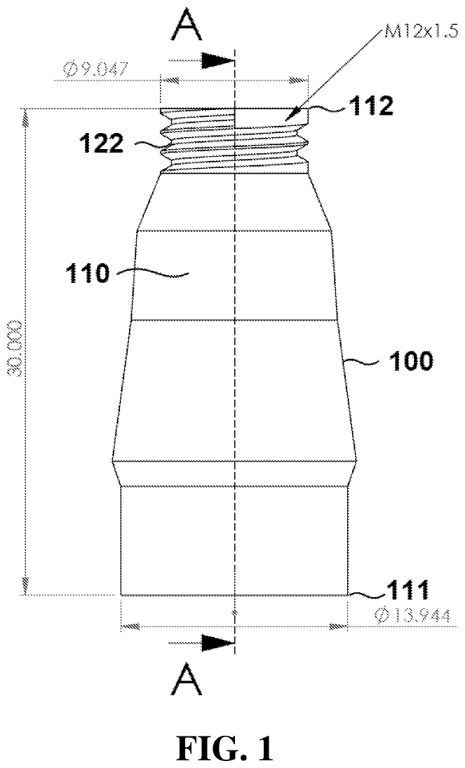

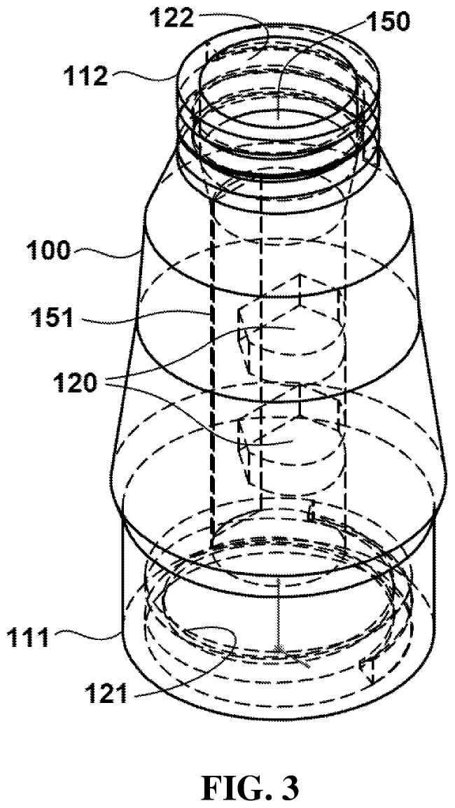

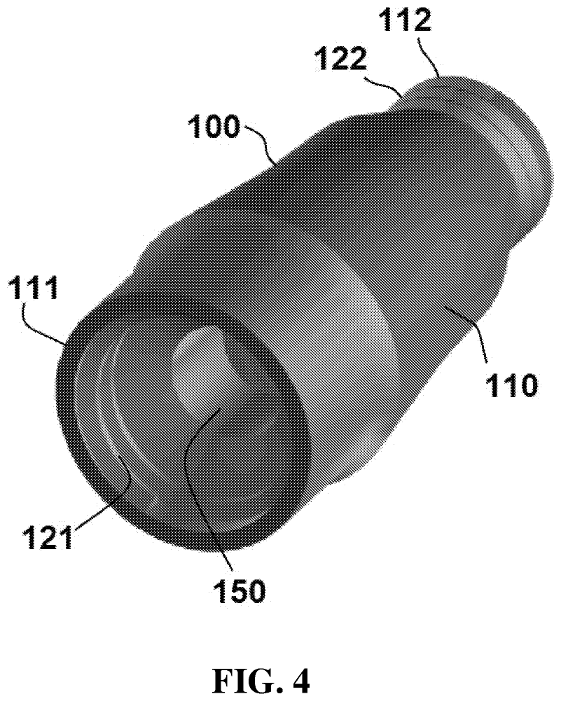

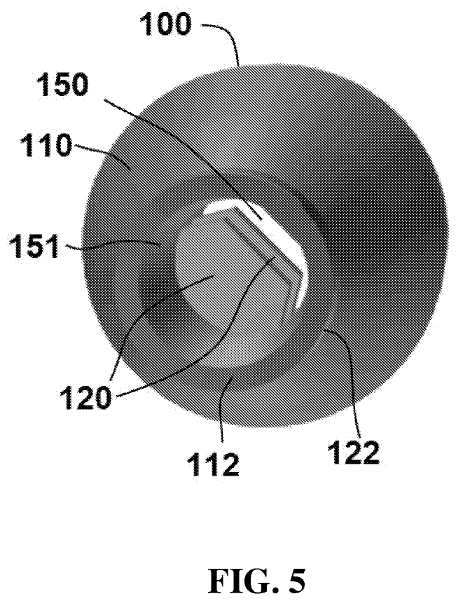

Referring initially to FIGS. 1-5, an exemplary embodiment of a device 100 for guidewire management is shown in orthographic, sectional and perspective views. While specific dimensions are provided in some of the illustrated figures, it is understood that exemplary embodiments of the present disclosure are not limited to the dimensions shown. Other embodiments may comprise dimensions different than those shown in the figures. For purposes of clarity, reference numbers for certain elements may not be shown in some of the figures.

As described in further detail below, device 100 is configured to be coupled to a distal end of a central line of a catheter. During use, device 100 can prevent a guidewire from inadvertently advancing into the patient during the insertion of the central line of the catheter.

In the embodiment shown, device 100 comprises a housing 110 with a first end 111 and a second end 112. Housing 100 comprises a central lumen 150 surrounded by a wall 151 that extends through housing 110 from first end 111 to second end 112. In addition, device 100 comprises one or more unidirectional valves 120 in central lumen 150. While the embodiment shown comprises two equivalently configured unidirectional valves 120, it is understood that other embodiments may comprise a single unidirectional valve 120 or more than two unidirectional valves 120. As previously noted, the term “valve” as used herein is not limited to devices or mechanisms that control or restrict fluids, and includes devices and mechanisms that can control or restrict movement of a solid (including for example, a guidewire) passing through the valve. In the embodiment shown, first end 111 comprises a female threaded coupling 121 and second end 112 comprises a male threaded coupling 122. In this embodiment, female threaded coupling 121 can be configured to couple to a central venous catheter. In addition, male threaded coupling 122 can be configured to couple to additional instruments coupled to the catheter if desired.

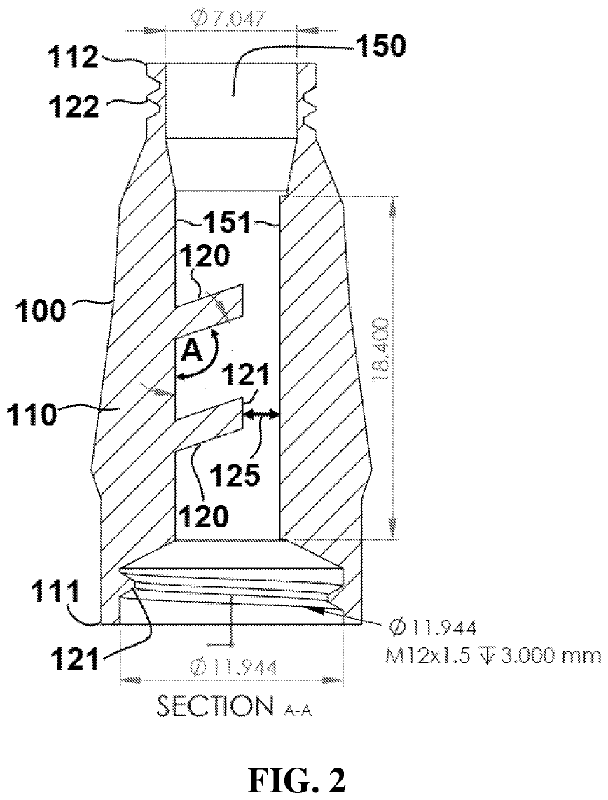

In the illustrated embodiment, unidirectional valves 120 are configured as flexible flaps that extend at an angle into central lumen 150. Unidirectional valves 120 are angled toward second end 112 to allow movement of a guidewire (not shown in FIG. 2) from first end 111 toward second end 112, but restrict movement of a guidewire from second end 112 toward first end 111. In particular embodiments, unidirectional valves 120 are angled from wall 151 toward second end 112 at an angle “A” as shown in FIG. 2. In certain embodiments, angle A between 100 and 120 degrees. In the specific embodiment shown in FIG. 2, unidirectional valves 120 are angled at approximately 109 degrees from wall 151. It is understood that other embodiments may comprise unidirectional valves 120 at a different angle than shown in the figures.

In the embodiment of FIG. 2, unidirectional valves 120 comprise a distal end 121 that is spaced apart from wall 151 by a gap 125. In particular embodiments, gap 125 is sized such that the maximum distance between distal end 121 and wall 151 is slightly smaller than the diameter of a guidewire (e.g. between 0.5 mm and 1.5 mm) that passes through aperture 150. Accordingly, as device 100 is moved relative to the guidewire, unidirectional valves 120 engage the guidewire. The angled positioning and flexible material of unidirectional valves 120 allow the guidewire to move in a direction from first end 111 toward second end 112 by further deflecting unidirectional valves 120 toward second end 112. However, the configuration of unidirectional valves 120 restrict movement of the guidewire in a direction from second end 112 toward first end 111. Attempted movement of a guidewire toward first end 111 would cause deflection of unidirectional valves 120 in a manner that would decrease gap 125. Accordingly, unidirectional valves 120 would further engage the guidewire and restrict movement of the guidewire in that direction.

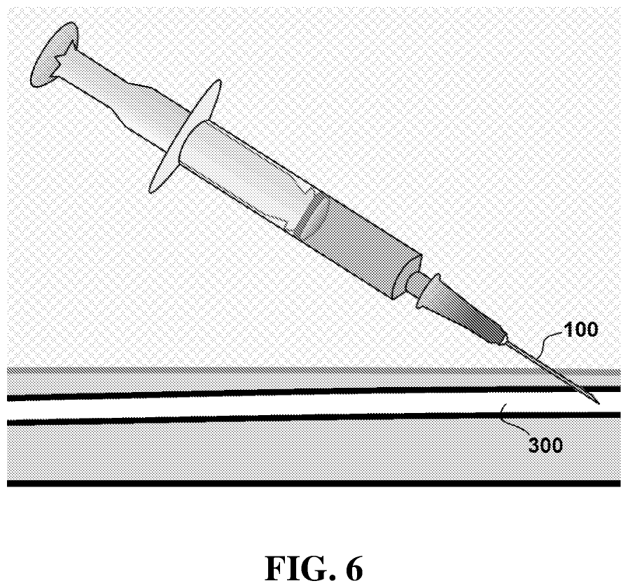

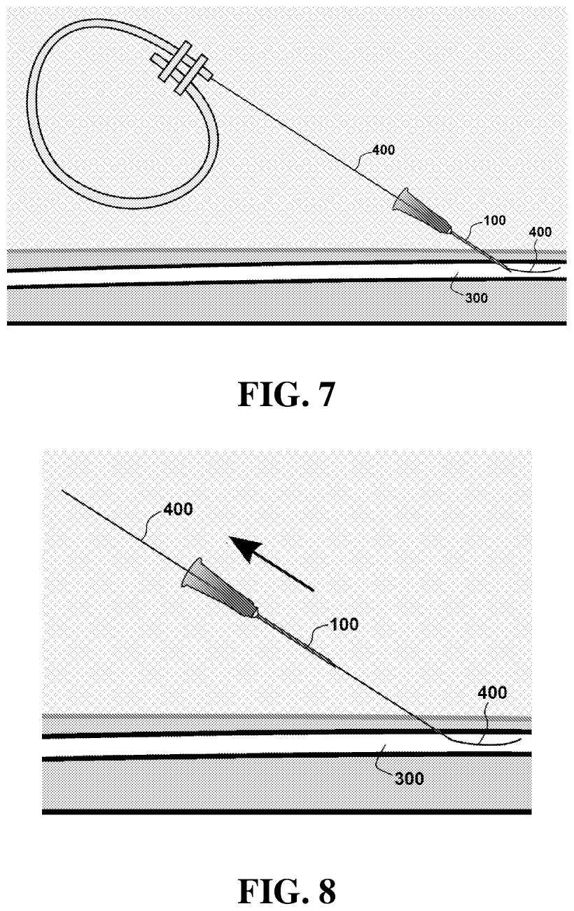

Referring now to FIGS. 6-10, multiple steps in a procedure utilizing device 100 are shown to allow retraction of a guidewire from a patient and restrict inadvertent further insertion of the guidewire into the patient. The steps illustrated in FIGS. 6-8 are typical for those used in guidewire insertion procedures, including for example, the Seldinger technique. In FIG. 6, a needle 200 is inserted into a vein 300 of a patient to provide access to the vein for the guidewire. In FIG. 7, a guidewire 400 is inserted through needle 200 into vein 300. Referring now to FIG. 8, the needle 200 is removed from guidewire 400 after guidewire 400 has been successfully inserted into vein 300. While the figures illustrate guidewire and catheter insertion into a vein, it is understood that other exemplary embodiments may include insertion into different organs or vessels of a patient.

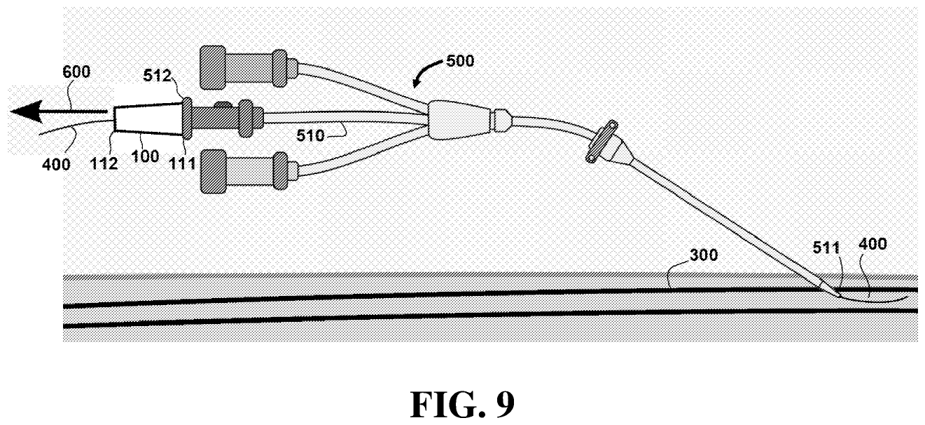

FIG. 9 illustrates a central venous catheter 500 with a proximal end 511 and a distal end 512. In the embodiment shown, device 100 is coupled to a distal end 512 of a central channel 510 of catheter 500. As shown in FIG. 9, catheter 500 (with device 100) is advanced along guidewire 400 until catheter 500 is inserted into vein 300. As previously described, unidirectional valves 120 (not visible in FIG. 9) allow catheter 500 and device 100 to advance along guidewire 400 so that catheter 500 can be inserted into vein 300. The relative movement of guidewire 400 with respect to catheter 500 and device 100 is shown by arrow 600. As shown, the relative movement of guidewire 400 with respect to device 100 is from first end 111 toward second end 112. However, unidirectional valves 120 restrict movement of guidewire 400 in a direction opposite of arrow 600 such that guidewire 400 is not permitted to move from second end 112 of device 100 toward first end 111. This restriction of movement of guidewire 400 can prevent guidewire 400 from being inadvertently inserted further into vessel 300 during use and potentially being retained in the patient.

If a user needs to move guidewire 400 in a direction opposite of arrow 600, the sides of device 100 can be depressed to allow free movement of guidewire 400. For example, housing 110 of device 100 can be formed from a flexible material that allows a user to compress (e.g. squeeze or pinch the outer portion of) device 100 in order to increase gap 125 (e.g. in a direction perpendicular to the plane of the section view shown in FIG. 2). The increased distance in gap 125 can allow guidewire 400 to freely move in either direction within device 100. In certain embodiments, device 100 is a unitary structure such that housing 110 and unidirectional valves 120 are formed from the same material. In exemplary embodiments, housing 110 and unidirectional valves 120 may be formed from any flexible medical grade material, including for example, polymer, latex, elastomer, silicone rubber, nylon, polyurethane, etc.

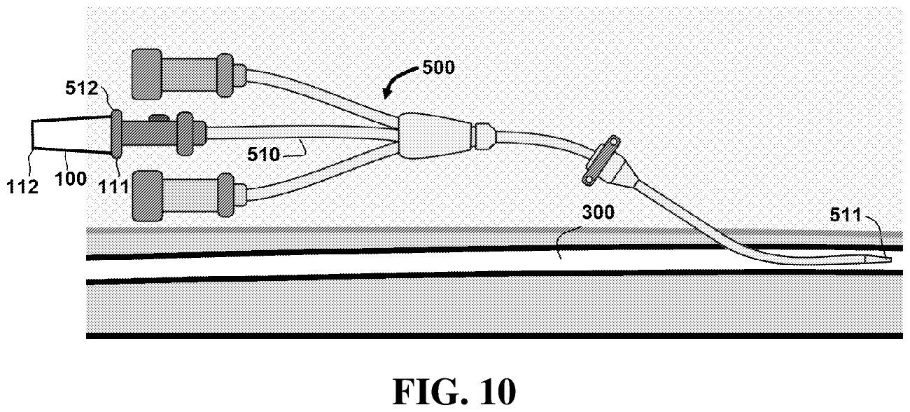

Referring now to FIG. 10, the guidewire has now been removed from catheter 500 and device 100. Device 100 can then be removed from catheter 500 and disposed of. Additional instruments can then be coupled to catheter 500 if desired and the procedure completed.

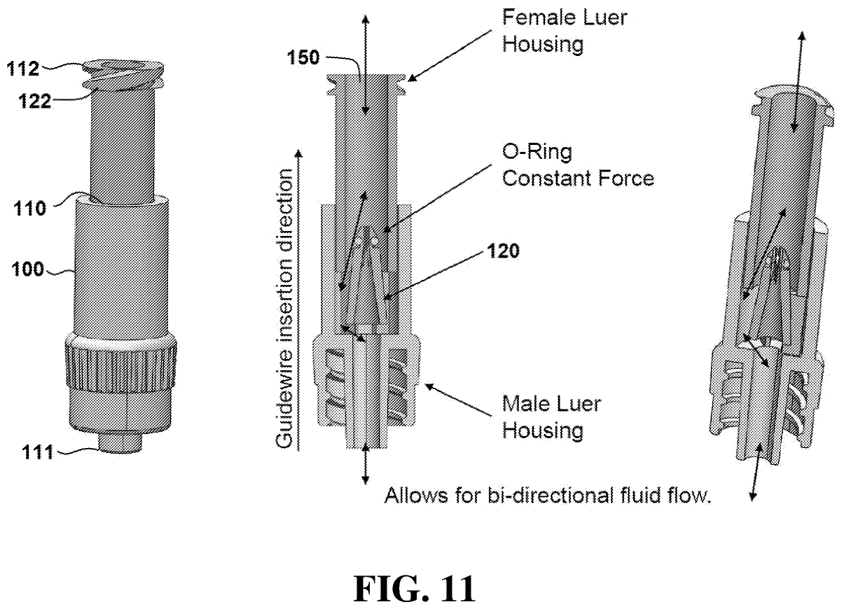

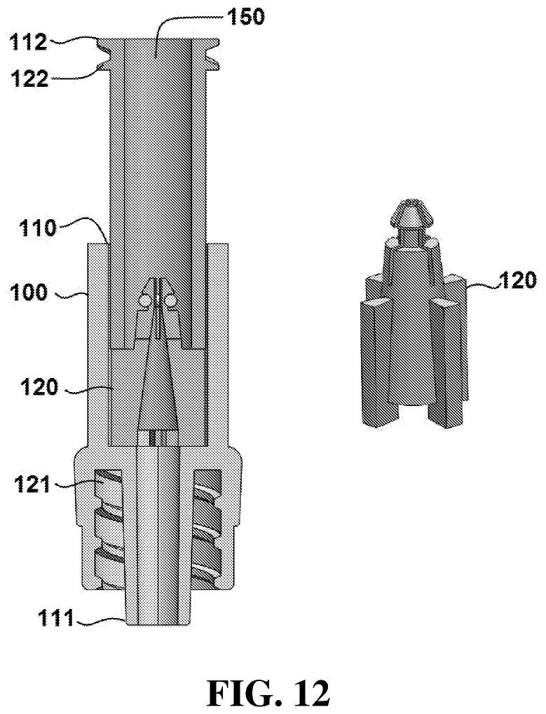

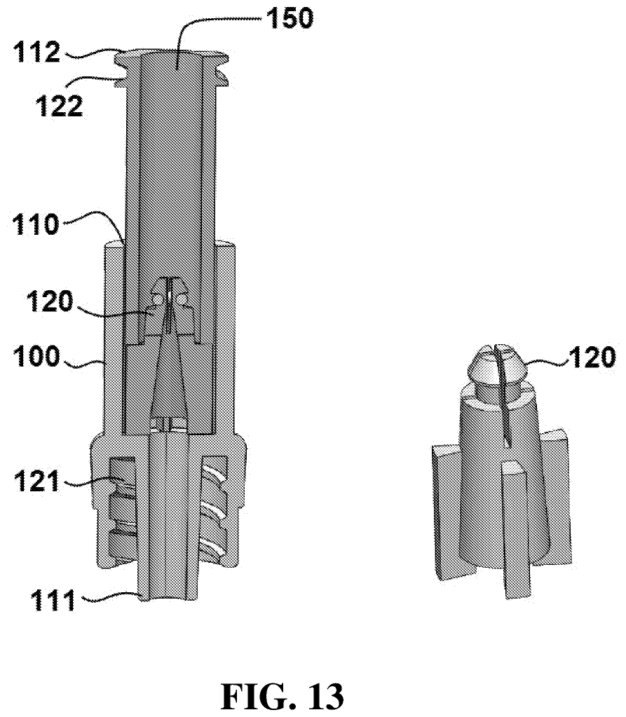

FIGS. 11-16 illustrate section and perspective views of additional embodiments of device 100. The principles of operation of the embodiments shown in FIGS. 11-16 are equivalent to the embodiment shown in FIGS. 1-10 and for sake of brevity will not be repeated here. As shown in the figures, each of the embodiments comprises housing 110 with first end 111 and second end 112. In the embodiments shown in FIGS. 11-16, housing 110 may comprise more than one component, including for example a female Luer housing and a male Luer housing.

In the illustrated embodiments, housing 100 also comprises central lumen 150 that extends through housing 110 from first end 111 to second end 112. In addition, the embodiments shown in FIGS. 11-16 comprise different configurations of unidirectional valve 120 in central lumen 150. In the embodiments shown, first end 111 comprises female threaded coupling 121, while second end 112 comprises a male threaded coupling 122. In this embodiment, female threaded coupling 121 can be configured to couple to a central venous catheter. In addition, male threaded coupling 122 can be configured to couple to additional instruments coupled to the catheter if desired.

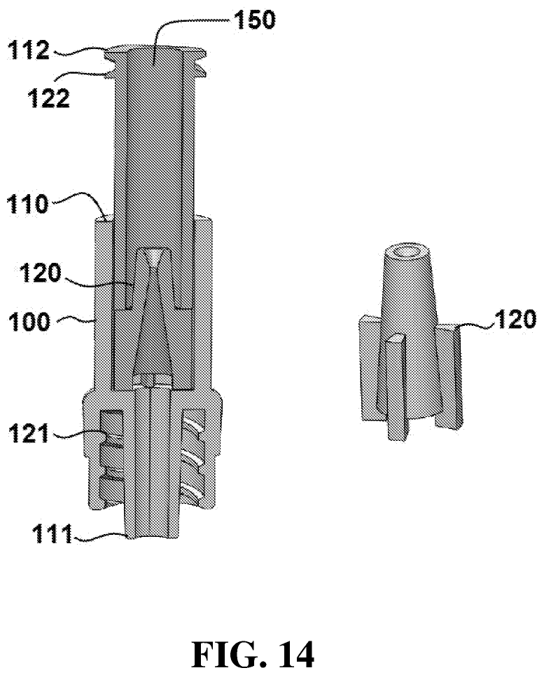

In the embodiments shown in FIGS. 11-13, unidirectional valve 120 comprises a tapered central lumen with cross-slits in the body of unidirectional valve 120 at the tapered end of the valve. In addition, the embodiments shown in FIGS. 11-13 may comprise a support member (e.g. an O-ring) surrounding the tapered end of the unidirectional valve 120. In the embodiment shown in FIG. 14, unidirectional valve 120 comprises a tapered central lumen with a reverse flare or reverse taper proximal to one end (e.g. the central lumen tapers for a first portion and then expands in diameter as one moves from one end of unidirectional valve 120 to a second end of unidirectional valve 120).

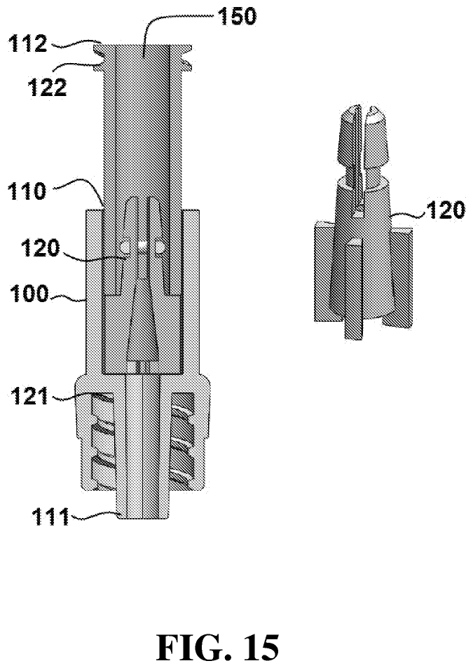

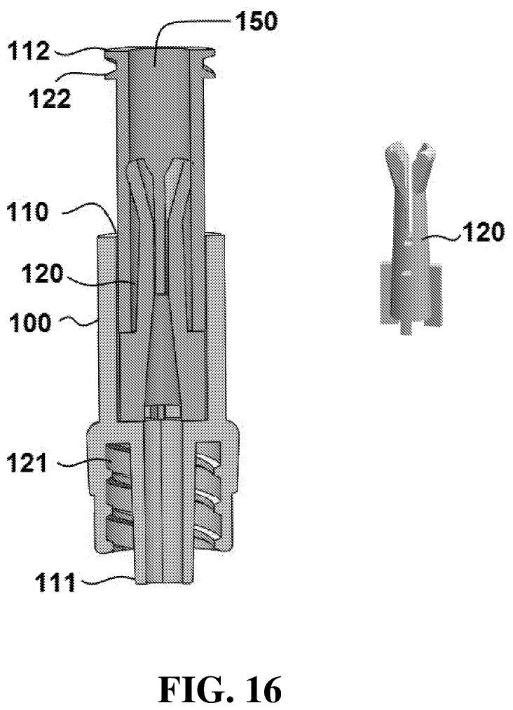

In the embodiment shown in FIG. 15, unidirectional valve 120 comprises a split portion proximal to one end. In addition, unidirectional valve 120 may comprise a support member (e.g. an O-ring) surrounding the split portion. In the embodiment shown in FIG. 16, unidirectional valve 120 comprises both a split portion and a tapered central lumen with a reverse flare or reverse taper proximal to one end (e.g. the central lumen tapers for a first portion and then expands in diameter as one moves from one end of unidirectional valve 120 to a second end of unidirectional valve 120). The split portion extends between the portions of the central lumen that include the taper and the reverse taper.

As shown and described herein, exemplary embodiments of the present disclosure provide devices and methods to provide for management of a guidewire during insertion of a catheter into a patient. Exemplary embodiments restrict unintentional advancement of a guidewire into the patient, reducing the likelihood of the guidewire inadvertently retained in the patient.

All of the apparatus, devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:
1. Seldinger S I (1953). “Catheter replacement of the needle in percutaneous arteriography; a new technique”. Acta Radiologica. 39 (5): 368-76. doi:10.3109/00016925309136722. PMID 13057644.
2. Schummer W, Schummer C, Gaser E, Bartunek R (2002). “Loss of the guide wire: mishap or blunder?”. British Journal of Anaesthesia. 88 (1): 144-6. doi:10.1093/bja/88.1.144. PMID 11881872.
3. McGee D C, Gould M K. Preventing complications of central venous catheterization. N Engl J Med 2003; 348:1123-33.
4. Vannucci A, Jeffcoat A, Ifune C, Salinas C, Duncan J R, Wall M. Retained guidewires after intraoperative placement of central venous catheters. Anesth Analg. 2013; 117(1):102-108.
5. Lin Y, Chou J, Chen Y, et al. A 20-year retained guidewire, should it be removed? Q J Med 2013; 106:373-374.

1. A device for guidewire management, the device comprising:
a housing comprising a first end and a second end;
a central lumen extending through the housing from the, wherein the central lumen extends from the first end to the second end; and
a unidirectional valve in the central lumen, wherein:
the unidirectional valve is configured to allow movement of a guidewire in a first direction through the unidirectional valve; and
the unidirectional valve is configured to prevent movement of a guidewire in a second direction through the unidirectional valve, wherein the second direction is opposite of the first direction.

2. The device of claim 1 further comprising:
a first threaded coupling in the central lumen, wherein the first threaded coupling is proximal to the first end; and
a second threaded coupling in the central lumen, wherein the second threaded coupling is proximal to the second end, wherein:
the first threaded coupling is configured to couple to a central venous catheter.

3. The device of claim 1 wherein the first direction is from the first end toward the second end.

4. The device of claim 1 further comprising a wall surrounding the central lumen, and wherein the unidirectional valve is angled from the wall toward the second end of the device.

5. The device of claim 4 wherein the unidirectional valve is angled from the wall at an angle between 100 and 120 degrees.

6. The device of claim 4 wherein the unidirectional valve is angled from the wall at an angle of approximately 109 degrees.

7. The device of claim 4 wherein:
the unidirectional valve comprises a distal end; and
the distal end is spaced apart from the wall by a gap.

8. The device of claim 7 wherein the gap is less than 0.5 mm.

9. The device of claim 1 wherein the device is a unitary structure and the housing and the unidirectional valve is formed from the same material.

10. The device of claim 9 wherein the material is a polymer.

11. The device of claim 9 wherein the material is an elastomer.

12. The device of claim 9 wherein the material is latex, silicone rubber, nylon, or polyurethane.

13. The device of claim 1 wherein the unidirectional valve is configured as a flexible flap.

14. The device of claim 1 wherein the unidirectional valve is a first unidirectional valve, and wherein the device further comprises a second unidirectional valve.

15. The device of claim 14, wherein the second unidirectional valve is configured equivalently to the first unidirectional valve.

16. A method of inserting a catheter into a patient, the method comprising:
inserting a guidewire into a patient;
obtaining a catheter comprising a proximal end and a distal end, wherein the catheter comprises a device according to claim 1 coupled to the distal end; and
advancing the catheter along the guidewire toward the patient, wherein the guidewire extends through the catheter and the device according to claim 1;
inserting the catheter into the patient; and
removing the guidewire from the device according to claim 1 and from the catheter.

17. The method of claim 16 wherein the guidewire and the catheter are inserted into a vein of the patient.

18. The method of claim 16 wherein the guidewire is restricted from moving from the distal end of the catheter toward the patient when the guidewire is inserted through the device according to claim 1.

19. The method of claim 18 further comprising:
compressing the device according to claim 1, wherein:
the guidewire is permitted to move from the distal end of the catheter toward the patient while the device according to claim 1 is compressed.

20. The method of claim 16 wherein the guidewire is inserted into the patient through a needle.

21. The method of claim 16 wherein the device according to claim 1 is coupled to the distal end of the catheter via a threaded coupling.

22. A system comprising:
a catheter comprising a proximal end and a distal end; and
a device coupled to the distal end, wherein:
the device is configured to allow movement of a guidewire in a first direction through the device and the catheter; and
the device is configured to prevent movement of a guidewire in a second direction through the device and the catheter, wherein the second direction is opposite of the first direction.

23. The system of claim 22 wherein the device coupled to the distal end comprises:
a housing comprising a first end and a second end;
a central lumen extending through the housing from the, wherein the central lumen extends from the first end to the second end; and
a unidirectional valve in the central lumen, wherein:
the unidirectional valve is configured to allow movement of a guidewire in a first direction through the unidirectional valve; and
the unidirectional valve is configured to prevent movement of a guidewire in a second direction through the unidirectional valve, wherein the second direction is opposite of the first direction.
